(19) United States
(12) Patent Application Publication
(10) Pub. No.: US 2026/0286558 A1
(43) Pub. Date: Sep. 24, 2026

(54) METHOD OF PRODUCING A HYDROGEN STREAM AND AN OXYGEN STREAM AND PASSING THE HYDROGEN STREAM AND THE OXYGEN STREAM TO A REVERSE WATER-GAS SHIFT REACTOR

(71) Applicant: JOHNSON MATTHEY DAVY TECHNOLOGIES LIMITED, London (GB)

(72) Inventors: Mohammad AHMADIANI, Stockton-on-Tees (GB); Henry Arthur CLAXTON, London (GB); Amelia Lorna Solveig COOK, London (GB); Elena Cristina CORBOS, Reading (GB); Hugh Gavin Charles HAMILTON, Reading Berkshire (GB)

(21) Appl. No.: 19/479,633

(22) PCT Filed: Apr. 11, 2024
(86) PCT No.: PCT/GB2024/050944

§ 371 Date: Oct. 28, 2025

(30) Foreign Application Priority Data
Jun. 7, 2023 (GB) 2308441.1

(51) Int. Cl. C25B 15/08; C01B 32/40; C10G 2/00; C25B 1/04; C25B 9/70

(52) U.S. Cl. CPC C25B 15/081; C01B 32/40; C10G 2/30; C25B 1/04; C25B 9/70

(57) ABSTRACT

A method of producing a hydrogen stream and an oxygen stream and passing the hydrogen stream and the oxygen stream to a reverse water-gas shift reactor is described, the method comprising: providing a water stream to an electrolysis system configured to form: a hydrogen stream at a first pressure, and an oxygen stream at a second pressure; passing the hydrogen stream, a carbon dioxide stream, and the oxygen stream to the reverse water-gas shift reactor, wherein the first pressure is lower than the second pressure.

### FIELD OF THE INVENTION

The invention relates to a method of producing a hydrogen stream and an oxygen stream and passing the hydrogen stream and the oxygen stream to a reverse water-gas shift reactor.

### BACKGROUND OF THE INVENTION

It is known to generate liquid hydrocarbons by performing a Fischer-Tropsch reaction on syngas comprising hydrogen and carbon monoxide. To increase the carbon monoxide content of the gas fed to the Fischer-Tropsch reaction to a favourable level, it is useful to subject a carbon dioxide gas stream to a reverse water-gas shift reaction with hydrogen to convert at least some of the carbon dioxide and hydrogen to carbon monoxide and water.

Reverse water-gas shift reactors are known. Such reactors comprise a reverse water-gas shift vessel containing a burner and optionally a fixed bed of reverse water-gas shift catalyst. The reactor is fed with a hydrogen, carbon dioxide and an oxygen stream that combusts a portion of the hydrogen, thereby generating heat for the endothermic reverse water-gas shift reaction.

WO2022079407A1 describes a method of synthesising hydrocarbons from syngas. The method involves the use of a water electrolyser to produce hydrogen and oxygen as feeds for a synthesis gas unit containing a reverse water-gas shift reactor. For every molecule of oxygen produced via electrolysis, two molecules of hydrogen are also produced. Typical reverse water-gas shift reactions use an excess of hydrogen compared to oxygen on a molar basis. The nature of reverse water-gas shift reactors means that the oxygen feed experiences a higher pressure drop when entering the reactor in comparison to the hydrogen feed. As a result, there is a need to supply the oxygen feed at a higher pressure than the hydrogen feed, typically by about 5 bara. This may be achieved by generating hydrogen and oxygen in the electrolyser at the lower desired hydrogen pressure and then using an oxygen compressor to increase the pressure of the oxygen to the higher desired oxygen pressure before the oxygen is passed to the reverse water-gas shift reactor. However, the use of an oxygen compressor increases the CAPEX and OPEX of the method and has safety implications. Alternatively, the hydrogen and oxygen may be generated in the electrolyser at the higher desired oxygen pressure with the hydrogen let down in pressure to the lower desired hydrogen pressure before being passed to the reverse water-gas shift reactor. However, having to let down the pressure of a large hydrogen flow rate decreases the efficiency of the method.

The present invention seeks to tackle at least some of the problems associated with the prior art or at least to provide a commercially acceptable alternative solution thereto.

### SUMMARY OF THE INVENTION

One aspect of the present disclosure is directed to a method of producing a hydrogen stream and an oxygen stream and passing the hydrogen stream and the oxygen stream to a reverse water-gas shift reactor, the method comprising:
providing a water stream to an electrolysis system configured to form:
a hydrogen stream at a first pressure, and
an oxygen stream at a second pressure;
passing the hydrogen stream, a carbon dioxide stream, and the oxygen stream to the reverse water-gas shift reactor,
   wherein the first pressure is lower than the second pressure.

### BRIEF DESCRIPTION OF THE DRAWINGS

FIG. 1 is a diagram of a first embodiment of a method according to the present invention.

FIG. 2 is a diagram of second and third embodiments of a method according to the present invention.

### DETAILED DESCRIPTION OF THE INVENTION

The present disclosure is directed to a method of producing a hydrogen stream and an oxygen stream and passing the hydrogen stream and the oxygen stream to a reverse water-gas shift reactor, the method comprising:
providing a water stream to an electrolysis system configured to form:
a hydrogen stream at a first pressure, and
an oxygen stream at a second pressure;
passing the hydrogen stream, a carbon dioxide stream, and the oxygen stream to the reverse water-gas shift reactor, wherein the first pressure is lower than the second pressure.

The method may be performed using an electrolysis system consisting of a single electrolysis unit comprising a single type of electrolyser that provides the hydrogen stream at the first pressure and the oxygen stream at the second pressure. The single type of electrolyser may comprise a plurality of electrolysing sub-units, each functioning in the same way, to perform the electrolysis. Preferably the method is performed using an electrolysis system comprising a first electrolysis unit and a second electrolysis unit, each electrolysis unit comprising one or more electrolysers. The invention therefore includes a method comprising:
providing a first water stream and a second water stream;
passing the first water stream to the first electrolysis unit to form a first hydrogen stream at the first pressure, and a first oxygen stream;
passing the second water stream to the second electrolysis unit to form a second hydrogen stream and a second oxygen stream at the second pressure;
combining the first hydrogen stream and the second hydrogen stream to form a combined hydrogen stream; and
passing the combined hydrogen stream, a carbon dioxide stream, and the second oxygen stream to the reverse water-gas shift reactor.

Each aspect or embodiment as defined herein may be combined with any other aspect(s) or embodiment(s) unless clearly indicated to the contrary. In particular, any features indicated as being preferred or advantageous may be combined with any other feature indicated as being preferred or advantageous.

Advantageously, the method of the present invention may avoid the need to compress the oxygen stream prior to passing it to the reverse water-gas shift reactor. This may improve the efficiency and safety of the method. In addition, there is no need to let down the pressure of the hydrogen stream prior to passing it to the reverse water-gas shift reactor. Accordingly, the efficiency of the method may increase.

Reverse water-gas shift reactors are known in the art. During the reverse water-gas shift reaction, carbon dioxide and hydrogen are converted as follows:

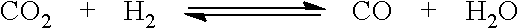

WO2022079408A1 discloses a typical reverse water-gas shift reactor. The reverse water-gas shift reactor typically converts a portion of the carbon dioxide and a portion of the hydrogen to carbon monoxide and water. As will be appreciated, not all of the hydrogen is converted. This is because of the need for the resulting syngas to contain hydrogen so as to undergo, for example, a subsequent Fischer-Tropsch reaction. A typical reverse water-gas shift reactor may comprise a refractory-lined long-necked swaged vessel. The feed gas typically enters the combustion region via a distributor to ensure uniform flow and hence uniform mixing. At the top of the vessel in the combustion region, oxygen is added via a burner nozzle into the hydrogen-rich environment. The long neck design and utilisation of a high momentum oxidant jet burner provides a mixed, combusted, uniform feed gas to the catalyst bed. This may ensure excellent reaction performance and physical stability of the catalyst. The high rates of mass and momentum transfer generates a relatively short, well-defined flame front that minimises radiation back onto the burner tip, thereby preventing it from overheating. The lower section of the reactor comprises a fixed catalyst bed where the endothermic reverse water-gas shift reaction occurs. This sits above a refractory dome often referred to as the “refractory arch”. The refractory arch is slotted to allow outlet gas to pass through and there is a catalyst underbed of alumina balls to avoid loss of catalyst through the refractory arch slots. Internal refractory lining is used to contain the high temperatures within the reactor and is installed into a supporting shell. The shell is encased in a boiler feed water jacket that maintains the shell temperature close to 100° C. by water vaporisation, which serves to remove any heat loss. Maintaining the shell temperature at 100° C. minimises thermal expansion and keeps the refractory in compression.

The method comprises providing a water stream to an electrolyser system. The water stream may be provided in the form of liquid water or steam.

In some arrangements the electrolysis system consists of a single electrolysis unit comprising a single type of electrolyser. In such arrangements, the single electrolysis unit may comprise an electrolyser having an anode producing the oxygen stream at a higher pressure than the cathode producing the hydrogen stream. However, this arrangement may generate excess pressurised oxygen that would require safe let down in pressure before being sent for use in other processes. In addition, a single electrolyser configuration may lead to more or larger equipment rated for the higher oxygen pressure leading to higher cost of fabrication. Accordingly, the electrolysis system preferably comprises a first electrolysis unit and a second electrolysis unit each electrolysis unit comprising one or more electrolysers, and the method comprises the steps of:
providing a first water stream and a second water stream;
passing the first water stream to the first electrolysis unit to form a first hydrogen stream at the first pressure, and a first oxygen stream;
passing the second water stream to the second electrolysis unit to form a second hydrogen stream, and a second oxygen stream at the second pressure;
combining the first hydrogen stream and the second hydrogen stream to form a combined hydrogen stream; and
passing the combined hydrogen stream, a carbon dioxide stream, and the second oxygen stream to the reverse water-gas shift reactor.

The method comprises providing one or more, preferably first and second, electrolysis units. Electrolysis units are known in the art. Electrolysis units typically comprise a single type of electrolyser and associated ancillary equipment. Suitable electrolysis units include, for example, alkaline electrolysers, polymer electrolyte membrane electrolysers, (including proton exchange membrane electrolysers and anion exchange membrane electrolysers—also known as alkaline exchange membranes electrolysers) and solid oxide electrolyser cell electrolysers, where the membrane can either be proton conducting or oxygen-ion conducting.

Where a single electrolysis unit is present, the hydrogen and oxygen may be generated at the required pressures for the reverse water-gas shift reactor using a single polymer electrolyte membrane electrolyser, such as a proton exchange membrane electrolyser or an anion exchange membrane electrolyser, operating in a differential pressure mode where the anode is operating at higher pressure than the cathode.

Where first and second electrolysis units are present, the first electrolysis unit and the second electrolysis unit may be of the same type. Alternatively, the first electrolysis unit and the second electrolysis unit may be of different types. In such arrangements, the pressure at the anode and cathode in the electrolysers may be the same or different, but the electrolysers are operated to provide the hydrogen and oxygen streams at the first and second pressures.

In an electrolysis unit, electricity is used to split water into hydrogen and oxygen, i.e.

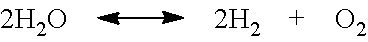

The electrolysis system preferably operates at temperatures from ambient to 1000° C. depending on the type of electrolysis unit used. For example, polymer electrolyte membrane electrolysers are typically used at a temperature in the range of 20-100° C., whereas solid oxide electrolyser cell electrolysers may be used at 400 to 1000° C., preferably from 450 to 900° C.

The method comprises passing the water stream to the electrolysis system to form a hydrogen stream at the first pressure, and an oxygen stream at the second pressure. To avoid the need to pressurise the hydrogen, the first pressure is preferably the pressure at which the hydrogen stream is to be passed to the reverse water-gas shift reactor.

In arrangements including the first and second electrolysis units, there are two hydrogen streams, which are combined, and first and second oxygen streams at different pressures. The first oxygen stream is not passed to the reverse water-gas shift reactor. This is because the first oxygen stream would need to be compressed prior to being passed to the reverse water-gas shift reactor, and the oxygen requirement is already met by the second oxygen stream that is provided at the higher second pressure. The first oxygen stream may be formed at the first pressure or at a pressure lower than the first pressure.

In arrangements including the first and second electrolysis units, the method comprises passing the second water stream to the second electrolysis unit to form a second hydrogen stream at a pressure which is at or higher than the first pressure and a second oxygen stream at the second pressure.

In arrangements including the first and second electrolysis units, the method comprises combining the first hydrogen stream and the second hydrogen stream to form a combined hydrogen stream. When the first pressure is the required pressure for hydrogen entry into the reverse water-gas shift reactor, the second hydrogen stream may be depressurised to the first pressure prior to combining the first hydrogen stream and the second hydrogen stream. This may ensure that the pressure of the combined hydrogen stream is a desired pressure for hydrogen entry into the reverse water-gas shift reactor. The second oxygen stream is fed to the reverse water-gas shift reactor. The first oxygen stream may be vented to the atmosphere or used outside of the process for combustion or as an oxidant in a separate chemical process that requires oxygen at a lower pressure.

Alternatively, when a singular electrolysis unit containing a single electrolyser type is used, the cathode in the electrolyser generates all of the hydrogen required at the first pressure for the reverse water-gas shift reactor and the anode generates oxygen at the second pressure required by the reverse water-gas shift reactor. The excess oxygen which is not sent to the reverse water-gas shift reactor may be used outside of the process for combustion or as an oxidant in a separate chemical process that requires oxygen or let down in pressure and sent outside battery limits.

The hydrogen stream or combined hydrogen stream fed to the reverse water-gas shift reactor is preferably subjected to purification in a hydrogen purification unit upstream of the reverse water-gas shift reactor.

The oxygen stream fed to the reverse water-gas shift reactor is preferably subjected to purification in an oxygen purification unit upstream of the reverse water-gas shift reactor.

Purification steps may be performed within the electrolysis system or electrolysis units downstream of the electrolyser or electrolysers to remove any water/steam and oxygen from the hydrogen steam and to remove water/steam and hydrogen from the oxygen stream to avoid potential explosive gas mixtures being passed downstream.

Alternatively, or in addition, contaminants may be removed from the hydrogen stream, and/or the oxygen stream in purification units located downstream of the electrolysis system or electrolysis units and upstream of the reverse water-gas shift reactor. The carbon dioxide stream fed to the reverse water-gas shift reactor may also be subjected to one or more stages of purification.

The method comprises passing the hydrogen and oxygen streams from the electrolysis system, and a carbon dioxide stream to a reverse water-gas shift reactor. The hydrogen stream from the electrolysis system is at a pressure suitable for entry into the reverse water-gas shift reactor.

The carbon dioxide stream may be mixed with the hydrogen stream from the electrolysis system or may be fed separately to the reverse water-gas shift reactor.

The first pressure of the hydrogen going to reverse water-gas shift reactor from the electrolysis system is lower than the second pressure of the oxygen going to the reverse water-gas shift reactor from the electrolysis system, for example at least 1 bar lower.

In a preferred embodiment, the reverse water-gas shift reactor is a catalysed reverse water-gas shift reactor. The use of a catalyst may increase the rate and/or yield and/or selectivity of the reverse water-gas shift reaction. In an alternative preferred embodiment, the reverse water-gas shift reactor is a non-catalysed reverse water-gas shift reactor.

Preferably, the reverse water-gas shift reactor comprises a reverse water-gas shift catalyst bed downstream of a burner; the hydrogen stream is delivered to the reverse water-gas shift reactor adjacent the burner and upstream of the reverse water-gas shift catalyst bed; and the oxygen stream is delivered to the burner. This may be a particularly effective arrangement for the reverse water-gas shift reactor. In addition, with such an arrangement the pressure drop experienced by the oxygen stream may be significantly greater than that experienced by the hydrogen stream.

In arrangements including the first and second electrolysis units, the amount of hydrogen contained in the first hydrogen stream passed to the reverse water-gas shift reactor is preferably from 1.1 to 12 times the amount of hydrogen contained in the second hydrogen stream passed to the reverse water-gas shift reactor on a volume or molar basis. For embodiments where the hydrogen from the second electrolyser is generated at a pressure greater than the first pressure, this will result in a smaller amount of hydrogen needing to be depressurised, thereby increasing the efficiency of the method compared to a single electrolyser system containing a singular electrolyser operating to provide a hydrogen and an oxygen stream at the second pressure.

The pressure of the hydrogen fed to reverse water-gas shift reactor from the electrolysis system, the first pressure, is lower than the pressure of the oxygen going to the reverse water-gas shift reactor, the second pressure. The first pressure is preferably lower than the second pressure by from 1 to 20 bar, more preferably from 2 to 8 bar. Such pressure differences are typical of the difference in depressurisations experienced by the hydrogen stream and the oxygen stream, respectively, on entry into the reverse water-gas shift reactor.

Preferably, the first pressure is from 10 to 55 bara, and/or the second pressure is from 11 to 60 bara. Such pressures are desirable for entry of the hydrogen stream and oxygen stream, respectively, into the reverse water-gas shift reactor.

In arrangements including the first and second electrolysis units, where the second hydrogen stream pressure is greater than the first pressure, the method preferably further comprises: depressurising the second hydrogen stream to the first pressure to form a depressurised second hydrogen stream; and introducing the depressurised second hydrogen stream into the first hydrogen stream to form the combined hydrogen stream before passing the combined hydrogen stream to the reverse water-gas shift reactor. This may ensure that the combined hydrogen stream is at a desirable pressure for entry into the reverse water-gas shift reactor.

The electrolysis system may comprise an alkaline electrolyser, a polymer electrolyte membrane electrolyser, or a solid oxide electrolysis cell electrolyser. In arrangements including the first and second electrolysis units, the first electrolysis unit and/or the second electrolysis unit preferably comprise an alkaline electrolyser, a polymer electrolyte membrane electrolyser, a solid oxide electrolysis cell, more preferably an alkaline electrolyser or a polymer electrolyte membrane electrolyser. Such electrolysers are known in the art. Such electrolysers are particularly suitable for forming hydrogen and oxygen streams at pressures suitable for entry into the reverse water-gas shift reactor.

In a preferred embodiment, the first and second electrolyser are both alkaline electrolysers where the first electrolyser generates hydrogen and oxygen at the first pressure and the second electrolyser generates oxygen and hydrogen at the second pressure.

In an alternative embodiment, the first and second electrolyser may be any combination of alkaline electrolyser, SOEC electrolyser, polymer electrolyte membrane electrolyser operating in balanced or differential mode. In this embodiment, the first electrolyser would generate hydrogen at the first pressure and the second electrolyser would generate oxygen at the second pressure. In one arrangement, the first electrolysis unit comprises a solid oxide electrolysis cell electrolyser and the first hydrogen stream is pressurised before being passed to the reverse water-gas shift reactor; and
the second electrolysis unit comprises a polymer electrolyte membrane electrolyser operating in balanced pressure or differential pressure mode, or an alkaline electrolyser.

Where a solid oxide electrolysis cell electrolyser is used, the produced hydrogen and oxygen gas streams may be above 450° C. and so may usefully be fed directly to the reverse water-gas shift reactor. If desired, the hydrogen and/or oxygen streams may be subjected, within or downstream of the electrolysis system or electrolysis units and upstream of the reverse water-gas shift reactor to one or more stages of cooling to below this temperature. The cooling may be performed by heating one or more process streams, for example by generating steam.

The electrolysis system or electrolysis units are preferably powered by renewable energy. This may render the method more environmentally friendly.

In a further aspect, the present invention provides a method of producing a hydrocarbon product, the method comprising:
producing a hydrogen stream and an oxygen stream and passing the hydrogen stream and the oxygen stream to a reverse water-gas shift reactor using the method described herein, wherein the hydrogen stream and oxygen stream are passed to the reverse water-gas shift reactor together with a carbon dioxide stream and form a carbon monoxide enriched syngas in the reverse water-gas shift reactor;
passing the carbon monoxide-enriched syngas to a Fischer-Tropsch unit to form a hydrocarbon product, a tail gas and an aqueous stream; and
recovering the hydrocarbon product.

The advantages and preferable features of the first aspect apply equally to this aspect.

The term “hydrocarbon product” has its usual meaning in the art. As used herein the term may encompass species formed of carbon and hydrogen, including those that are liquid at room temperature and pressure. The hydrocarbon product typically comprises alkanes, and typically comprise from 5 to 100, or higher, carbon atoms per molecule. The present invention is particularly suitable in processes for the synthesis of liquid hydrocarbon fuels such as gasoline, diesel and kerosene.

In the method, the hydrogen stream, oxygen stream and a carbon dioxide stream are all passed to the reverse water-gas shift reactor. The carbon dioxide stream may be any carbon dioxide stream. The carbon dioxide may be recovered from combustion gases, or the carbon dioxide may be contained within or separated from process streams such as a synthesis gas stream or biogas streams. The carbon dioxide may also be recovered from air by direct air capture or recovered from sea water. The carbon dioxide stream may comprise minor amounts of species other than carbon dioxide, for example carbon monoxide, hydrogen and water.

The method comprises passing the carbon monoxide-enriched syngas to a Fischer-Tropsch unit to form a hydrocarbon product, a tail gas and an aqueous stream. Fischer-Tropsch reactors are known in the art. The temperature of the Fischer-Tropsch reactor is preferably from 150° C. to 300° C. Lower temperatures may result in unfavourably low levels of hydrocarbon product being generated. Higher temperatures may increase the energy cost of the method without a significant increase in the levels of hydrocarbon product being produced and risks thermal damage to the catalyst. The Fischer-Tropsch reactor preferably comprises a catalyst comprising cobalt, iron and/or ruthenium. Such a catalyst may be particularly effective at catalysing Fischer-Tropsch reactions and/or enable the reaction to proceed at favourably low temperatures and/or with high yield. The molar ratio of hydrogen to carbon monoxide in the syngas is preferably from 1.8 to 2.2 since this is close to the stoichiometric ratio of the Fischer-Tropsch reaction of about 2.

The tail gas may comprise one or more of C1 to C4 hydrocarbons, water and unreacted species such as hydrogen, carbon monoxide and carbon dioxide.

The Fischer-Tropsch unit may include a downstream upgrading unit in which the hydrocarbon product from the Fischer-Tropsch reactor is converted, typically by hydroprocessing using hydrogen gas, into useful liquid hydrocarbon products, such as kerosene, diesel and naphtha with by-product liquid petroleum gas (LPG) and non-condensable off-gases.

The aqueous stream produced in the Fischer-Tropsch unit may, after purification, be recycled to the first water stream and/or second water stream. Alternatively or additionally, process condensate recovered from synthesis gas downstream of the reverse water-gas shift reactor may, after purification, be recycled to the electrolysis system. This may reduce the amount of water required by the method. The aqueous streams are preferably at least partially purified prior to being passed to the electrolysis system. For example, hydrocarbons, ammonia and dissolved gases may be removed from the aqueous stream. Impurities, such a hydrocarbons, may clog the electrolysis units and/or poison catalysts contained in the cathode and/or anode of the electrolysis units.

### Examples

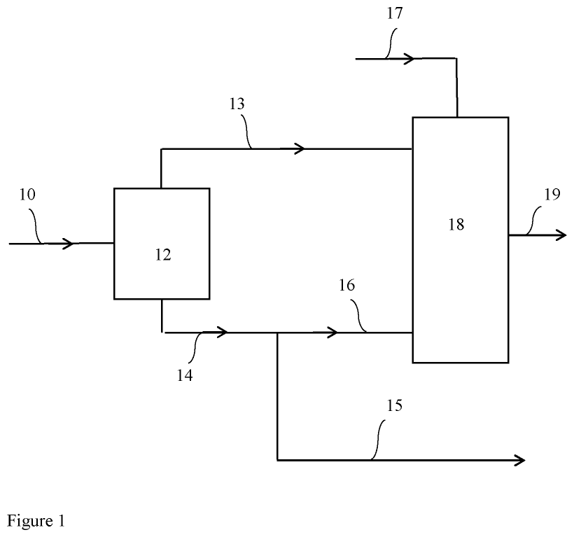

FIG. 1 shows a diagram of a first embodiment of a method according to the present invention. A single water stream 10 is passed to an electrolysis system consisting of a single electrolysis unit containing a single electrolyser type 12, which forms a hydrogen stream 13 at the cathode at a pressure of 25 bar and a flow rate of 300 kmol/h, and also an oxygen stream 14 at the anode at a pressure of 30 bar and a flow rate of 150 kmol/h. The oxygen stream 14 is split into stream 15 with a flow rate of 120 kmol/h and stream 16 with a flow rate of 30 kmol/h. This single electrolyser unit comprises a polymer membrane electrolyte electrolyser operating in differential mode with the anode operating at a pressure greater than the cathode. Hydrogen stream 13, oxygen stream 16, and carbon dioxide stream 17 are passed to a reverse water-gas shift reactor 18. Oxygen stream 15 may undergo depressurisation (upstream or downstream of the balance of plant units) and is passed to outside battery limits. In the reverse water-gas shift reactor 18, a portion of the hydrogen 13 is combusted with the oxygen 16 and the hot gas mixture containing the carbon dioxide 17 is passed through a bed of reverse water-gas shift catalyst to form a synthesis gas comprising carbon monoxide and hydrogen, which is recovered from the reactor 18 via line 19. The synthesis gas from line 19 is converted to hydrocarbon products in a Fischer-Tropsch unit (not shown).

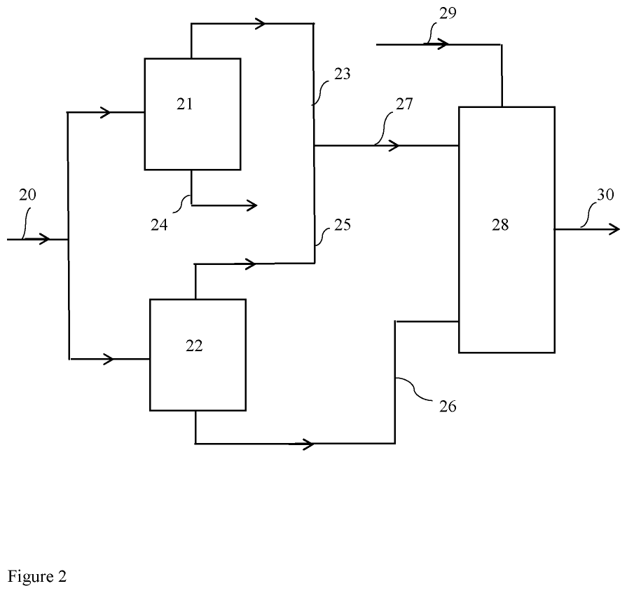

FIG. 2 depicts second and third embodiments of a method according to the present invention using first and second electrolysis units. In the second embodiment a water stream 20 is divided and fed to a first electrolysis unit 21 and a second electrolysis unit 22. The first electrolysis unit 21 forms a hydrogen stream 23 at a pressure of 25 bar and a flow rate of 240 kmol/h, and an oxygen stream 24 at a pressure of 25 bar and flow rate of 120 kmol/h. The second electrolysis unit 22 forms another hydrogen stream 25 at a pressure of 30 bar and a flow rate of 60 kmol/h, and another oxygen stream 26 at a pressure of 30 bar and a flow rate of 30 kmol/h. The electrolyser units 21 and 22 each comprise one or more alkaline electrolysers. The hydrogen stream 25 from electrolysis unit 22 is depressurised to 25 bar (upstream or downstream of the balance of plant units) and combined with the hydrogen stream 23 from electrolysis unit 21 to form a combined hydrogen stream 27, which is passed to the reverse water-gas shift reactor 28. Oxygen stream 24 from electrolysis unit 21 may undergo depressurisation (upstream or downstream of the balance of plant units) and is passed to outside battery limits. Oxygen stream 26 from electrolysis unit 22 is passed to the reverse water-gas shift reactor 28. A carbon dioxide stream 29 is also fed to the reverse water-gas shift reactor. In the reverse water-gas shift reactor 28, a portion of the combined hydrogen stream 27 is combusted with the oxygen 26 and the hot gas mixture containing the carbon dioxide 29 is passed through a bed of reverse water-gas shift catalyst to form a synthesis gas comprising carbon monoxide and hydrogen, which is recovered from the reactor 28 via line 30. The synthesis gas from line 30 is converted to hydrocarbon products in a Fischer-Tropsch unit (not shown).

The third embodiment differs from the second embodiment in that the electrolysis unit 21 comprises an alkaline electrolyser, a polymer electrolyte membrane operating in balanced or differential mode, or a solid oxide electrolysis cell electrolyser. Where a solid oxide electrolysis cell electrolyser is used it may be that hydrogen stream 23 could require an extra stage of compression before it is combined with hydrogen stream 25. If a polymer electrolyte membrane electrolyser is used in differential mode with the cathode operating at a pressure greater than the anode, the oxygen stream 24 may not require depressurisation. In addition, electrolysis unit 22 may comprise an alkaline electrolyser or a polymer electrolyte membrane operating in balanced mode or differential where the anode operates at a pressure greater than the cathode. If a polymer electrolyte membrane electrolyser is used in differential mode with the anode operating at a pressure greater than the cathode, the hydrogen stream 25 may not require depressurisation prior to being combined with hydrogen stream 23.

The foregoing detailed description has been provided by way of explanation and illustration and is not intended to limit the scope of the appended claims. Many variations in the presently preferred embodiments illustrated herein will be apparent to one of ordinary skill in the art and remain within the scope of the appended claims and their equivalents.

1. A method of producing a hydrogen stream and an oxygen stream and passing the hydrogen stream and the oxygen stream to a reverse water-gas shift reactor, the method comprising:
providing a water stream to an electrolysis system configured to form:
a hydrogen stream at a first pressure, and
an oxygen stream at a second pressure;
passing the hydrogen stream, a carbon dioxide stream, and the oxygen stream to the reverse water-gas shift reactor,
wherein the first pressure is lower than the second pressure.

2. The method according to claim 1, wherein the electrolysis system comprises a single electrolysis unit containing a single type of electrolyser having an anode producing the oxygen stream at a higher pressure than the cathode producing the hydrogen stream.

3. The method according to claim 1, wherein the electrolysis system comprises a first electrolysis unit and a second electrolysis unit each electrolysis unit comprising one or more electrolysers, comprising:
providing a first water stream and a second water stream;
passing the first water stream to the first electrolysis unit to form a first hydrogen stream at the first pressure, and a first oxygen stream;
passing the second water stream to the second electrolysis unit to form a second hydrogen stream, and a second oxygen stream at the second pressure;
combining the first hydrogen stream and the second hydrogen stream to form a combined hydrogen stream; and
passing the combined hydrogen stream, a carbon dioxide stream, and the second oxygen stream to the reverse water-gas shift reactor.

4. The method of claim 1, wherein the reverse water-gas shift reactor is a catalysed or non-catalysed reverse water-gas shift reactor.

5. The method of claim 1, wherein:
the reverse water-gas shift reactor comprises a reverse water-gas shift catalyst bed downstream of a burner;
the hydrogen stream is delivered to the reverse water-gas shift reactor adjacent the burner and upstream of the reverse water-gas shift catalyst bed; and
the oxygen stream is delivered to the burner.

6. The method of claim 3, wherein the amount of hydrogen contained in the first hydrogen stream passed to the reverse water-gas shift reactor is from 1.1 to 12 times the amount of hydrogen contained in the second hydrogen stream passed to the reverse water-gas shift reactor.

7. The method of claim 1, wherein the first pressure is lower than the second pressure by from 1 to 20 bar, preferably from 2 to 8 bar.

8. The method of claim 1, wherein, the first pressure is from 10 to 55 bara, and/or the second pressure is from 11 to 60 bara.

9. The method of claim 3, where the second hydrogen stream pressure is greater than the first pressure further comprising:
depressurising the second hydrogen stream to the first pressure to form a depressurised second hydrogen stream; and
introducing the depressurised second hydrogen stream into the first hydrogen stream to form the combined hydrogen stream before passing the combined hydrogen stream to the reverse water-gas shift reactor.

10. The method of claim 1, wherein a hydrogen stream is fed to a hydrogen purification unit upstream of the reverse water-gas shift reactor.

11. The method of claim 1, wherein an oxygen stream is fed to an oxygen purification unit upstream of the reverse water-gas shift reactor.

12. The method of claim 1, wherein the electrolysis system comprises an alkaline electrolyser, a polymer electrolyte membrane electrolyser, or a solid oxide electrolysis cell electrolyser, preferably an alkaline electrolyser or a polymer electrolyte membrane electrolyser.

13. The method of claim 3, wherein the first electrolysis unit and the second electrolysis unit each comprise an alkaline electrolyser.

14. The method of claim 3, wherein the first and/or second electrolysis units comprise an alkaline electrolyser or a polymer electrolyte membrane electrolyser operating in a balanced pressure mode or a differential pressure mode.

15. The method of claim 1, wherein the oxygen stream is not compressed prior to being passed to the reverse water-gas shift reactor.

16. The method of claim 3, wherein:
the first electrolysis unit comprises a solid oxide electrolysis cell electrolyser and wherein the first hydrogen stream is pressurised before being passed to the reverse water-gas shift reactor; and
the second electrolysis unit comprises a polymer electrolyte membrane electrolyser operating in balanced pressure or differential pressure mode, or an alkaline electrolyser.

17. The method of claim 1, wherein the electrolysis system is powered by renewable energy.

18. The method of claim 1, wherein process condensate recovered from a synthesis gas downstream of the reverse water-gas shift reactor is purified and recycled to the electrolysis system.

19. A method of producing a hydrocarbon product, the method comprising:
producing a hydrogen stream and an oxygen stream and passing the hydrogen stream and the oxygen stream to a reverse water-gas shift reactor using the method of claim 1 to form a carbon monoxide enriched syngas in the reverse water-gas shift reactor;
passing the carbon monoxide-enriched syngas to a Fischer-Tropsch unit to form a hydrocarbon product, a tail gas and an aqueous stream; and
recovering the hydrocarbon product.

20. The method of claim 19, wherein the aqueous stream produced in the Fischer-Tropsch unit is purified and recycled to the electrolysis system.
